(19) United States
(12) Patent Application Publication
(10) Pub. No.: US 2003/0222247 A1
(43) Pub. Date: Dec. 4, 2003

(54) Methods for manufacturing luminescent products having long afterglow

(76) Inventors: Everly Dean Putman, Davenport, IA (US); Gary Leroy Butler, Atlanta, GA (US)

Correspondence Address: SUTHERLAND ASBILL & BRENNAN LLP, 999 PEACHTREE STREET, N.E., ATLANTA, GA, 30309, US

(21) Appl. No.: 10/352,480

(22) Filed: Jan. 28, 2003

Related U.S. Application Data
(60) Provisional application No. 60/351,734, filed on Jan. 28, 2002.

(51) Int. Cl. C09K 11/02; A61K 7/04

(52) U.S. Cl. 252/301.36; 106/31.32; 106/31.64; 424/61; 523/105; 428/690

(57) ABSTRACT

Method of manufacturing a luminescent product exhibiting a daytime color and a glow in the dark color, where the glow in the dark color exhibits long term glow in the dark characteristics after absorbing energy from light sources. At least one resin material and at least one antisettling agent are mixed and combined with a plurality of luminescent crystals capable of glowing in the dark after the absorption of light. This mixture is combined with solvents, colorants, and additives to produce a luminescent material having a daytime color that differs from its glow-in-the-dark color.

### RELATED APPLICATION DATA

The present application claims the benefit and priority of U.S. Provisional Application No. 60/351,734, titled “Color Luminescence Chemical Process For Cloth Material Products”, filed on Jan. 28, 2002, the entire contents of which are incorporated herein by reference.

### FIELD OF THE INVENTION

The present invention relates generally to luminescent products, and more specifically, to methods for manufacturing products which exhibit a daytime color and a glow in the dark color, where the glow in the dark color exhibits long term glow in the dark characteristics after absorbing energy from light sources such as sunlight, fluorescent lights and incandescent lights.

### BACKGROUND OF THE INVENTION

Photostorage materials such as phosphorescent materials are those that can absorb energy when excited by solar, fluorescent and other artificial lights. In particular, when these materials are excited by ultraviolet light, they deviate from their initial equilibrium states, and convert the absorbed energy to visible light after they return to the equilibrium states so as to continue to glow after the excitation has stopped. These materials are advantageous for use in a multitude of products that benefit from enhanced visibility in the dark, including household and recreational items, such as paints, clothing, material, toys, cosmetics and the like, as well as outdoor articles such as paint, road signs, decals, and safety equipment.

Luminous paints have been applied on items such as watch dials and safe marks. These paints typically exhibit a yellow-green glow and have usually been formed by admixing a photostorage or phosphorescent material, using zinc sulfide mixed with copper as an activator, into paint, ink and the like. The sulfides can absorb energy when excited by ultraviolet light with certain wavelengths and then release the energy in the form of visible light in accordance with the above light-emitting principle of the phosphorescent material. Since the sulfides, however, have a very short span of afterglow, have unstable chemical structures and are not lightproof, there exist many problems when they are used in a practical situation. For example, the visible light emitted by such materials can last only for 20-30 minutes when they are used for luminous watches. There may also be the phenomenon of light decomposition and even loss of light-emitting ability of the material when it is radiated with ultraviolet radiation. Therefore, they can not be used in outdoor environments.

To prolong the time of afterglow, radioactive materials, such as Pm, are sometimes added to give phophors light-emitting ability. However, with radioactive materials the requirement for the treatment of the materials is very strict, and high costs arise for the apparatus used and the treatment of waste materials such as waste water, so this method is not used at present. Besides sulfide phosphorescent material, it has been suggested that photostorage materials be prepared by adding the rare earth element europium to alkaline earth metal aluminates. For example, U.S. Pat. No. 3,294,699, to Lange, discloses a light-emitting material of strontium aluminate in which divalent Eu is utilized as an activator and the amount added is 2-8 mol % of strontium aluminate. This fluorescent material has a light-emitting peak of 520 nm when excited by ultraviolet light. Unfortunately, however, because this fluorescent material has little afterglow it is not suitable for use in products where a long after-glow is preferred. Additionally, this material can be problematic to use in cosmetic and other products which might come into regular and prolonged contact with skin, which could be aggravated by the phosphor composition. Furthermore, these materials are limited in their glow in the dark colors to a yellow-green glow so that they may not be suitable for a variety of applications where a specific glow in the dark color is desired.

U.S. Patent No. 5,885,483 suggests the use of a synthetic super luminescent crystal composed of aluminum oxide, strontium oxide, calcium oxide, europium oxide and boron oxide to produce luminescent products. This unique luminescent material accumulates light for high initial brightness and long afterglow duration. It absorbs light from the sun, fluorescent lights, and other light sources that excite it and glows for up to 10 hours. This product offers excellent resistance to environmental conditions, including sunlight, superior chemical resistance, and a long product life of 10 years or more. Unfortunately, this material is only available in two colors that glow in the dark—green glow and blue glow. Additionally, both are light yellow-green in day light conditions.

U.S. Pat. No. 6,177,029 discloses the use of a photostorage and emissive material having a broad range of color options. The material features high initial brightness and prolonged decay time while. The material also has daytime brightness and night time glow in the dark properties with a wide range of colors. According to the patent, one and/or two or more different fluorescent colorant and/or optical brightener materials could be combined with the luminescent material in order to create a wide range of photostorage and emissive materials of different colors. This allows the creation of custom colors for manufacturing products which provide a unique glow color appearance for improved product recognition and marketability.

Although the material described by the '029 patent provides a unique glow and color appearance, and may be used in paints, printing inks, and the like, its application to numerous commercial products can produce undesirable results in terms of texture and appearance. Additionally, the durability of commercial products, and the capacity of the products to glow in the dark for long durations, may be compromised based upon the process used to apply such a photostorage material. Therefore, what is needed are methods for manufacturing a variety of products that exhibit a daytime color and a glow in the dark color, where the glow in the dark color exhibits long term glow in the dark characteristics after absorbing energy from light sources.

### BRIEF SUMMARY OF THE INVENTION

The present invention overcomes the disadvantages of the prior art by providing methods for manufacturing a variety of products that exhibit a daytime color and a glow in the dark color, where the glow in the dark color exhibits long term glow in the dark characteristics after absorbing energy from light sources. Products that may be produced using the present invention include luminescent: household and recreational items, such as paints, clothing, material, toys, cosmetics (e.g., nail polish, makeup, etc.) and the like, as well as outdoor articles such as paints, road signs, decals, and safety equipment.

According to one embodiment of the present invention, there is disclosed a method of manufacturing a luminescent product that exhibits a daytime color and a glow in the dark color, where the glow in the dark color exhibits long term glow in the dark characteristics after absorbing energy from light sources. The method includes the steps of mixing at least one resin material and at least one antisettling agent to produce a resin material and antisettling agent mixture, and adding at least one luminescent crystal to the resin material and antisettling agent mixture to produce a resin and luminescent crystal mixture. The method further includes the step of mixing at least one solvent with at least one colorant to produce a solvent and colorant mixture, and combining the resin and luminescent crystal mixture with the solvent and colorant mixture, and at least one additive, to produce a luminescent material, wherein the luminescent material exhibits a daytime color and a glow-in-the-dark color.

According to one aspect of the present invention, the daytime color differs from the glow-in-the-dark color. According to another aspect of the invention, the step of combining the resin and luminescent crystal mixture with the solvent and colorant mixture and at least one additive includes the step of combining the resin and luminescent crystal mixture with the solvent and colorant mixture and at least one additive to produce a luminescent material, wherein the luminescent material includes a paint or die. Additionally, according to the invention, the at least one additive can include a plurality of glass microspheres that provide an insulting effect to the luminescent material so that the luminescent material can include an insulating paint or dye. According to yet another aspect of the invention, the step of combining the resin and luminescent crystal mixture with the solvent and colorant mixture and at least one additive includes the step of combining the resin and luminescent crystal mixture with the solvent and colorant mixture and at least one additive to produce a luminescent material, wherein the luminescent material includes luminescent tape.

Furthermore, according to the invention, the step of combining the resin and luminescent crystal mixture with the solvent and colorant mixture and at least one additive includes the step of combining the resin and luminescent crystal mixture with the solvent and colorant mixture and at least one additive to produce a luminescent material, wherein the luminescent material includes at least 50%, by volume, of the at least one resin material. The step of mixing the at least one resin material and at least one antisettling agent can also include the step of mixing the at least one resin material and at least one antisettling agent using high speed dispersion or a static mixer. Moreover, the method can further include the step of encapsulating the at least one luminescent crystal prior to the step of adding the at least one luminescent crystal to the resin material and antisettling agent mixture to produce a resin and luminescent crystal mixture.

According to yet another aspect of the present invention, the method includes the step of applying the luminescent material to an item to produce a luminescent product. According to a further aspect of the invention, the step of applying the luminescent material to the item includes using at least one applicator to apply the luminescent material, wherein the at least one applicator is selected from the group consisting of: a paint brush, a foam brush, an airless sprayer, a paint gun, a screen printer, and a dot jet printer. The luminescent material may also be heated after the luminescent material is applied to the item using the at least one applicator. Furthermore, the item may be primed with a base material, such as with a white-colored base material, prior to the application of the luminescent material.

According to another embodiment of the present invention, there is disclosed a luminescent product that exhibits a daytime color and a glow in the dark color, where the glow in the dark color exhibits long term glow in the dark characteristics after absorbing energy from light sources. The product includes a luminescent material comprising at least one resin material, at least one antisettling agent, at least one colorant, at least one additive, and at least one luminescent crystal, where the luminescent material exhibits a daytime color and a glow-in-the-dark color, and where the daytime color differs from said glow-in-the-dark color. According to one aspect of the invention, the product further includes at least one base material in contact with the luminescent material. According to another aspect of the invention, the product comprises a product selected from the list of products consisting of: nail polish, paint, fabric paint, dyes, reflective tape, road markers, road signs, outlet covers, lamp shades and apparel. According to yet another aspect of the invention, the luminescent material comprises a plurality of microspheres, wherein said plurality of microspheres provide an insulting effect to the luminescent material, where the luminescent material can comprises insulating paint.

### BRIEF DESCRIPTION OF THE SEVERAL VIEWS OF THE DRAWING(S)

Having thus described the invention in general terms, reference will now be made to the accompanying drawings, which are not necessarily drawn to scale, and wherein:

FIG. 1 shows a block diagram illustrating the components comprising a luminescent material for use in manufacturing luminescent products, according to one embodiment of the present invention.

FIG. 2 shows a block diagram flow chart illustrating the process of creating a luminescent material for use in manufacturing luminescent products, according to one embodiment of the present invention.

FIG. 3 shows a block diagram flow chart illustrating a process for applying the luminescent material to a commercial product, according to multiple aspects of the present invention.

### DETAILED DESCRIPTION OF THE INVENTION

The present inventions now will be described more fully hereinafter with reference to the accompanying drawings, in which some, but not all embodiments of the invention are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

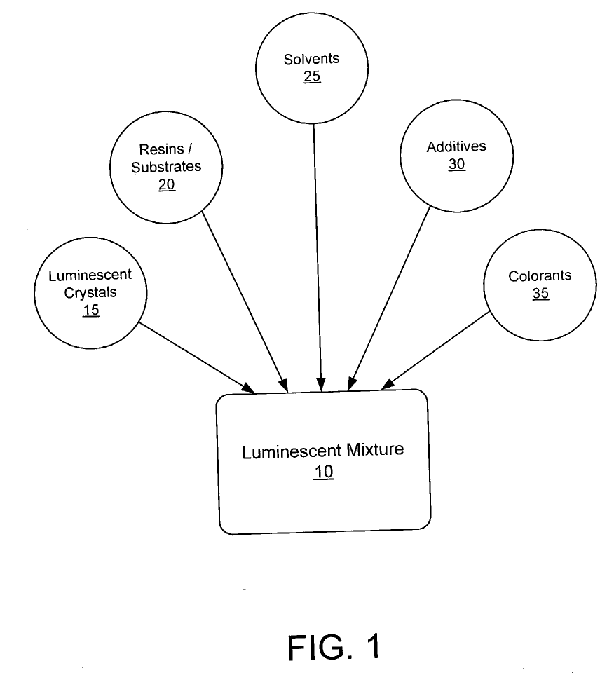

FIG. 1 shows a block diagram illustrating the components comprising a luminescent material 10 for use in manufacturing luminescent products, according to one embodiment of the present invention. According to the present invention, the luminescent material 10 includes one or more: luminescent crystals 15, resins/substrates 20, solvents 25, additives 30 and colorants 35. The luminescent crystals 15 provide the luminescent material 10 and extended night time glow so as to enable products manufactured therewith to provide high initial glow-in-the-dark brightness and a prolonged light-emission decay time. The crystals are a photostorage and emissive material having a broad range of glow in the dark colors. According to one aspect of the invention, the crystals 15 may comprise synthetic Aluminum Oxide crystals as disclosed in U.S. Pat. No. 6,177,029, the contents of which is hereby incorporated herein in its entirety. According to another aspect of the invention, Strontium Aluminate crystals may be used. Other luminescent materials composed of one or more of aluminum oxide, strontium oxide, calcium oxide, europium oxide and boron oxide, and intermixes thereof may also be used. The use of these luminescent crystals 15 allows the creation of custom colors for manufacturing products which provide a unique glow color appearance for improved product recognition and marketability. As explained in detail below, the present invention utilizes the multi-color glow-in-the-dark capability of the crystals 15 to manufacture products having a first, daytime color and a second, glow in the dark color. This allows for outstanding flexibility in the creation of products for virtually any industry.

Generally, the greater amount of crystals inserted into the luminescent material 10, the greater the capacity of the mixture to capture and re-emit light. However, because the crystals 15 are coarse, a high concentration of crystals 15 generally causes the luminescent material 10 to be uneven or bumpy. Therefore, the volume of crystals placed in the mixture 10 is typically kept at or below 30% of the entire volume of the luminescent material 10. However, it will be appreciated that a higher concentration of crystals may be used where the luminescent material 10 is applied to a product requiring a higher glow irrespective of the smoothness of the mixture finish. Similarly, lower volumes of crystals 15 may be employed in the mixture 10 where a highly viscous mixture 10 is desired, as in the case of a mixture to be applied via spraying.

According to one aspect of the invention, because the luminescent crystals 15 include rough surfaces, they may be coated prior to insertion into the luminescent material 10. This may be accomplished by gravity feeding the crystals through glass or clear coating substance which allows the crystals to emit light while preventing the rough edges from causing undesirable consequences in an end product. For instance, where the luminescent material 10 is used to produce glow-in-the-dark cosmetics, the smooth edges will prevent the crystals 15 from aggravating sensitive skin. Another solution of the present invention is smoothing the edges of the crystals 15 prior to their inclusion into the luminescent material 10. This may be accomplished by grinding, cutting, sanding, or otherwise altering the crystals 15 physically to eliminate their sharp edges.

Referring again to the components of the luminescent material 10 illustrated in FIG. 1, the resins/substrates 20 provide the luminescent material 10 molecular integrity and a base for the luminescent crystals 15 and the other ingredients of the mixture 10. According to one aspect of the invention, the resins/substrates 20 compose the largest portion of the luminescent material 10. As will be appreciated by those of skill in the art, resins/substrates 20 bind together the ingredients in the luminescent material 10, provide adhesion to the surface upon which the mixture 10 is applied, and provide the mixture 10 with its coating properties, including gloss, durability and resistance. Typically, resins/substrates 20 start out as liquids but dry to form tough durable films. For instance, where the luminescent material 10 represents luminescent paint, the resins/substrates 20 start out as a liquid, and are applied as such. However, as such luminescent paint is applied and dries, the paint forms a thin-film in the same manner as conventional paints. Of course, because the paint includes luminescent crystals 15 infused therein, the paint can glow in the dark.

The properties of the resins/substrates 20, which make up approximately 50% of the luminescent material, largely determine the properties of the material 10. Resin and substrate materials which are suitable for use in the present invention include those materials provided in Table 1, below.

TABLE 1
Resins and Substrates

| Synthetic Resins: | Natural Resins: | Other Substrates |
|---|---|---|
| Acrylic - | Sandarac | East India Kino - Malabar |
| Polypropenonitrile, | Guaiacum | Kino, Kino Gum |
| Polymethyl Methacrylate | Storax Enamel | |
| Polyethylene | Scammony | Polymer - Copolymer, |
| Allyl Resin | Acaroid Resin | Polyurethane, Lignin, |
| Urea-formaldehyde Resin | Animc | Polyamide, Trimer |
| Alkyd | Asaftida | Plastic - ABS plastic, Mylar, |
| Phenolic Resin | Camphor | Thermoplastic, Bakelite, |
| Epoxy | Cannabin | Teflon, Vinylite, Resinoid, |
| Melamine Resin | Amber | Amino plastic, Cellulosic, |
| Polyester | Copal - Courbaril copal, | Coumarone-indene resin, |
| Vinyl Polymer - | Copalite, Congo copal, | Fluorocarbon plastic, |
| Polyvinyl Acetate, | Kauri, Zanzibar copal | Phenolic plastic, Polyester, |
| Polyvinyl Chloride, | Dammar | Polypropylene, Polyvinyl- |
| Styrene | Colophony | formaldehyde, silicone |
| | Mastic | resin, vinyl, Thermosetting |
| | Oleoresin - Labdanum, | compositions |
| | Balsam, Canada Balsam, | |
| | Turpentine, Capaiba | |
| | Gum Resin - Elemi, Myrrh, | |
| | Sonora Gum, Benzoin, | |
| | Bdellium, Gamboge, | |
| | Myrrh | |
| | Wood Tar | |

As evidenced by the table, there are a wide variety of resins and substrates which may be used to in the luminescent material 10. According to one aspect of the invention, one or more of the above resins, or like resins not listed in the above table, may be combined in the luminescent material 10. Because the properties of the resins/substrates, which make up approximately 50% of the luminescent material, largely determine the properties of the material 10, the resins/substrates 20, or combinations thereof, are selected based upon the end-use of the luminescent material 10.

The resins/substrates 20 are thinned with one or more solvents 25, which are liquids that typically thin and maintain a consistent viscosity of the resin/substrate 20. Because the solvents 25 adjust the viscosity of the resin/substrate 20, they regulate the viscosity of the luminescent material 10. Therefore, the amount and type of solvent(s) 25 selected are critical in ensuring that the mixture 10 will can be applied to an end product. In a preferred embodiment, the solvents 25 typically comprises approximately 10% of the volume of the luminescent material 10, though higher volumes of solvents 25 may be used to thin or thicken the luminescent material 10. A non-exhaustive list of solvents utilizable in the present invention is provided in Table 2, below.

TABLE 2
Solvents

| | | |
|---|---|---|
| Alcohols | n-Amyl acetate | Diisobutyl ketone |
| Methyl alcohol | Butyl lactate | Cyclohexanone |
| Ethyl alcohol | Propylene glycol | Isophorone |
| n-Propyl alcohol | monoethyl ether acetate | Diacetone alcohol |
| Isopropyl alcohol | Methyl amyl acetate | Methyl amyl ketone |
| Isoamyl alcohol | Diethyl ether | Acetonitrile |
| Cyclohexanol | Diisopropyl ether | Nitromethane |
| Ethylene glycol | Tetrahydrofuran | Nitroethane |
| Glycerol | Cellosolve″solvent² | Castor oil |
| Formamide | Toluene | Linseed oil |
| Dimethyl | Xylene | Soya |
| formamide | | |
| Methylene chloride | n-Hexane | Fatty Acid |
| Ethylene dichloride | Cyclohexane | Vegetable Oil |
| Perchloroethylene | VM£¦P naphtha | Chlorinated Solvents |
| 1,1,1-Tricholoro- | Mineral spirits | Oxygenated Solvents |
| ethane | | |
| Methyl fomate | Turpentine | Oxides and Glycols |
| Ethyl acetate | Acetone | Water borne, e.g., H₂O |
| Isopropyl acetate | Methyl ethyl ketone | Chlorinated Solvents |
| n-Butyl acetate | Methyl isobutyl ketone | Oxygenated Solvents |
| | | Oxides and Glycols |

The above list, though extensive, is intended to be illustrative only, and thus is not intended to limit the types of solvents, known to those of skill in the art, which are suitable for adding to the resins/substrates 20. Furthermore, like the resins/substrates 20, a plurality of solvents 25 may be combined to achieve a particular mixture and consistency of the luminescent material 10 so that it may be used in manufacturing luminescent end products.

Next, additives 30 are incorporated into the luminescent material 10 to impart a variety of properties into the mixture 10 including flow, stability, drying, defoaming, mildew resistance and viscosity. Additives 30 include anti-skinning agents, used to prevent undesirable surface drying, or skinning, in containers during storage and shipment. They also include pigment suspending aids, dispersants, and surfactants which impart stability and promote shelf-life. Furthermore, additives include viscosity and flow enhancers, which ensure that the luminescent material 10 will provide desired application properties. Additives 30 also ensure appropriate characteristics such as gloss and texture, and that the luminescent material 10 includes specific coating performance properties. Those additives listed in Table 3 represent some of the additives that may be included in the luminescent material 10 of the present invention. Like the above material listings, Table 3 represents only illustrative materials and is not intended to be an exhaustive list.

TABLE 3
Additives

Additives for the crystal and pigments

Wetting agent
Dispersing agent
Anti-floating agent

Surface additives

Flow agents
Leveling agents
Defoamers
De-airaters

Rheology additives

Rheology modifiers
Anti-setting agents

Other

Anti-skinning agents
Preservers
Driers
Adhesion Promoters
Catalysts
Release agents

According to one aspect of the invention, glass or ceramic microspheres can constitute an additive 30 inserted into the luminescent material 10. The microspheres provide an insulating effect to the material 10. Therefore, where the luminescent material 10 is an interior or exterior paint, the microspheres can effectively provide an “R” value that will effectively reduce heating and cooling costs.

Finally, colorants 35 (or pigments) are employed to provide the daytime color of the luminescent material 10. The colorants 35 may comprise titanium dioxide (a white pigment), colored organic or inorganic pigments, carbon black pigments, or a similar colorants, as known in the art, for dying the luminescent material 10 a variety of colors. Preferably, the colorants 35 used are capable of being used in a multitude of applications, including in cosmetics, soaps and food products. These pigments 35 can include inert pigments such as calcium carbonate (limestone), talc, clay, sand (silica). All of these are naturally occurring materials that are tightly bound in the mixture 10 after it is applied, and contribute various properties like gloss control and flow.

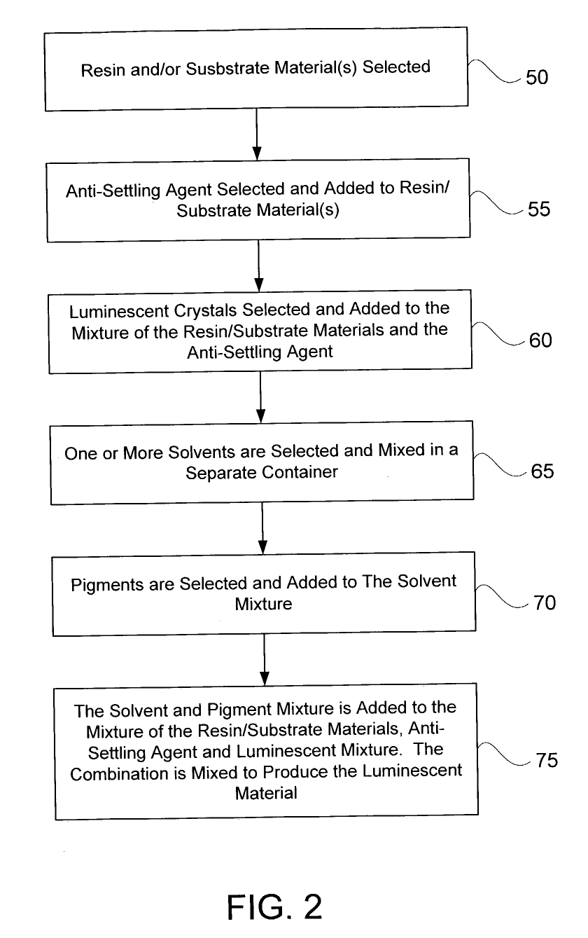

The selection of and methods for combining these materials 15, 20, 25, 30 and 35 is illustrated in the block flow diagram of FIG. 2, which illustrates the process of creating a luminescent material 10 for use in manufacturing luminescent products. Initially, the process begins with the selection of a resin or substrate material (block 50), or combination thereof. The resins/substrate materials are chosen to bind the components of the luminescent material 10 together, and can make up between 1 and 95% of the luminescent material 10, though 50% is preferred in most applications. As noted above, and illustrated in the examples below, the resins/substrates are preferably chosen to match the desired qualities of the luminescent material 10 created by the methods of the present invention.

After resins and/or substrates are selected, they are mixed together using high speed dispersion or a static mixer. High speed dispersion is relatively inexpensive, but inserts air into the mixture, which is undesirable because it can affect the properties of the mixture and cause the temperature of the mixture to vary, which can impact the viscosity of the materials. On the other hand, static mixing is preferred because it introduces the least amount of air into the mixture. Additionally, when used to mix the final luminescent material 10, static mixing limits damage to the crystals. These crystals 15 would otherwise be damaged if more violent mixing procedures were used, such as paint grinding.

After the resins/substrates 20 are chosen and combined, if necessary, an antisettling agent (an additive material) is added to the resin (block 55). The antisettling agent typically comprises between 1-10% of the luminescent material 10. The antisettling agent is an additive that is mixed with the resins/substrates to prevent caking or hard sedimentation of crystal and pigments, in the luminescent material 10. Next, the luminescent crystals 15 are then added to this mixture (block 60). Typically, the luminescent crystals comprise 5 to 80% of the total weight of the luminescent material 10, with the material 10 consisting of approximately 30%, total volume, of crystals 15 for most end applications. The crystals are chosen based upon the desired glow-in-the-dark color of the luminescent material 10.

Next, one or more solvents 25 are selected and mixed together in container separate from the resin/antisettling agent/crystal mix (block 65). The one or more solvents 25 typically constitute 1 to 90% of the luminescent material 10, though 10% is preferred where a high viscosity is unnecessary. This is the case, e.g., where the luminescent material 10 is used as finger nail polish or as interior or exterior paint. However, a higher volume of solvents 25 are required where high viscosity is necessary. This would be necessary, for instance, where the luminescent material 10 is used as ink or is applied via a paint gun or sprayer (e.g., an airless sprayer).

After the one or more solvents 25 are selected and mixed together in a separate container, the pigments (or colorants) are added thereto (block 70). The pigments 35 are added to provide color to the luminescent material 10. The color of the pigments 35 are visible in lit conditions, as provided by light sources such as sunlight, fluorescent lights and incandescent lights. Therefore, the pigments 35 provide the day time color of the luminescent material 10, while the crystals provide the luminescent, glow-in-the-dark color of the material 10. The allows products made by the present invention to exhibit two different colors—one during the daytime to make a product visually appealing, and a second, glow-in-the-dark color to make the product stand out or satisfy a marketing requirement.

Finally, the solvent and pigment mixture is added to the mixture of the resins/substrate, antisettling agent, and luminescent crystal (block 75). The materials are mixed using mixing techniques discussed in detail above. Also added are any additional additives 30 (other than the antisettling agent, added earlier) identified in Table 3, above. Along with the antisettling agent, these additional additives typically constitute approximately 1 to 10% of the total weight of the luminescent material 10. However, glass or ceramic microspheres, if added, can constitute a large percentage of the total volume of the luminescent material 20, such as 50%.

Although the process described above is the preferred embodiment for mixing the materials that comprise the luminescent material 10, it will be appreciated that other methods may be used. For instance, all of the materials may be simultaneously added and mixed. Likewise, the materials may also be added to a single container in succession and mixed one or more times during such a process. Next, the following three examples provide illustrative mixtures of each component of the luminescent material 10 for various applications.

### EXAMPLE 1

Luminescent Interior Wall Paint

Luminescent Crystal:

25 grams.

Resins/Substrates:

Polyurethane, 46.4 grams; and

Acrylic, 25 grams.

Solvents:

Water, 5 grams;

Dipropylene Glycol n-Butyl Ether (DPnB), 3 grams; and

Dipropylene Glycol Methyl Ether (DPM), 1 gram.

Additives:

Leveling agent, 0.2 grams;

Antisettling agent, 5 grams;

Antifoaming agent, 1 gram; and

Glass Microsphere 6 grams.

Pigment/Colorant:

Color dependent for amount.

In this illustrative example, luminescent crystals, which provide luminous emissions in the dark, are bound in two resins/substrates. In particular, polyurethane was selected due to its flexibility, long life and resistance to chemicals and marring. This was combined with Acrylic, which accentuates the polyurethane and provide enhanced adhesion, a hard surface and relatively fast dry time, each of which are important for paint products. The resin/substrate combination is flexible, water soluble, resistant to ware, low odor, fast dry time, optical clarity, and overall appearance.

Next, water, DPnB and DPM were selected as solvents. Respectively, these materials reduce the thickness of the resins/substrates, provide a slow evaporating speed to control dry time, and prevent the coagulation of polyurethane. The additives were also selected with the end product, wall paint, in mind. The leveling agent allows for brush strokes to level out, the antisettling agent prevents caking or hard sedimentation of crystal and pigments, and the antifoaming agent takes the air out of the mixture to prevent imperfections in coatings. Additionally, glass microspheres were added to help disperse light from the luminescent crystals in an even manner. Finally, the pigment and colorant is product dependent. It provides the daytime color of the interior wall paint. Generally, any pigment or colorant that is translucent and provides color fastness is desirable.

### EXAMPLE 2

Luminescent Nail Polish

Luminescent Crystal:

25 grams

Resins/Substrates:

Polyurethane 75 grams.

Solvents:

Water, 10 grams;

DPnB, 4 grams.

Additives:

Leveling agent, 0.2 grams;

Antisettling agent, 3 grams;

Antifoaming agent, 1 gram;

Glass Microsphere, 10 grams, and

Thickening Agent, 0.3 grams.

Pigment/Colorant:

Color dependent for amount.

In this example the base polyurethane resin was chosen because it is extremely fast in the time it takes to set from application. It will be appreciated that this short “wet to set time” is advantageous for nail polish. Additionally, the polyurethane resin provides excellent flexibility and resistance to chemicals and marring. Therefore, the life of the nail polish is extended to 1-2 weeks, in comparison to conventional polish, which may only last 2-4 days without chipping and marring. Additionally, the resin is relatively easy to remove. The above formula allows polish to be removed without the use of a thinner, which can leave a residual staining on nails and skin. Rather, the material peals off when so desired, leaving no residue of color or material. It also has low odor.

As with the interior paint example, DPnB was selected as a solvent because it provide a slow evaporating speed to control dry time. The additives were also selected with the product, luminescent nail polish, in mind. The leveling agent allows for brush strokes to level out, the antisettling agent prevents caking or hard sedimentation of crystal and pigments, and the antifoaming agent takes the air out of the mixture to prevent imperfections in coatings. Additionally, glass microspheres were added to help disperse light from the luminescent crystals in an even manner and a thickening agent was used to increase the thickness of coating so that multiple nail polish coats are not required. Finally, the pigment and colorant is product dependent. It provides the daytime color of the interior wall paint. Generally, any pigment or colorant that is translucent and provides color fastness is desirable.

### EXAMPLE 3

Luminescent Exterior Enamel

Luminescent Crystal:

20 grams

Resins/Substrates:

Long oil Alkyd, 37 grams.

Solvents:

Mineral Spirits, 12.6 grams;

Additives:

Rheology modifier, 1 gram;

Antisettling agent, 5 grams;

Calcium Drier, 0.6 grams;

Zirconium Drier, 0.4 grams;

Cobalt Drier, 0.3 grams;

Anti-Skinning Agent, 0.2 grams;

Defoamer, 0.2 grams; and

Glass Microsphere, 5 grams.

Pigment/Colorant:

Color dependent for amount.

In this example the base resin, a Long oil Alkyd, was chosen for its flexibility and abundance in coatings industry. The resin is a general purpose coating that is relatively inexpensive to produce while providing good flexibility and a slow dry time. Additionally, the resin provides slow wear, which is important for exterior applications. The solvent, Mineral Spirits, is used to reduce and disperse the resin. Next, a series of additives are included to thicken the solution (rheology modifier), prevent caking (antisettling agent), to cure the Alkyd (Calcium Drier, Zirconium Drier, and Cobalt Drier), and to prevent or retard the oxidation or polymerization which results in the formation of an insoluble skin (Anti-skinning Agent). Other additives include a defoamer and glass microsphere, which perform the same functions identified in the previous examples.

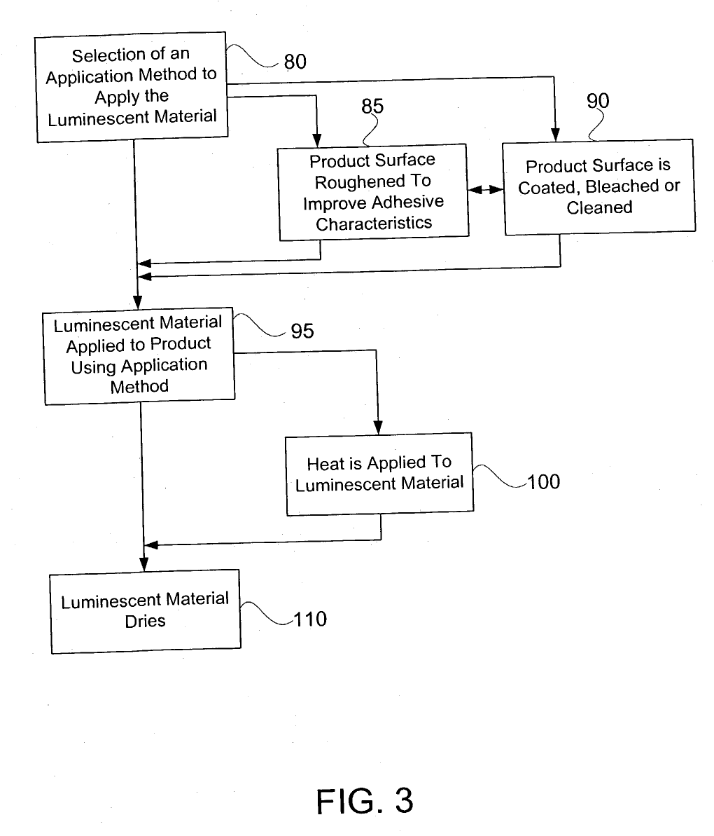

Referring now to FIG. 3, after the luminescent mixture 10 is prepared, it is ready to be applied to a luminescent product. Application methods for applying the luminescent mixture include: rolling on, paint brushing, foam brushing, airless spraying, air fed paint gun, air brush, dip method, screen print, dot jet printed, offset printing, lithograph, sputtering, and gravity fed dispersion. According to one aspect of the invention, a luminescent product surface need not be prepared prior to application of the luminescent material 10. Therefore, in many circumstances, the luminescent material 10 can be applied using one or more of the above application methods directly to a product (blocks 80, 95). For instance, where the luminescent material is viscous, it may be applied directly to, e.g., apparel or textiles using a sprayer. Similarly, where the luminescent material has the consistency of paint it may be applied directly to a wall or surface (e.g., an automobile, helmet, or similar article to be painted) without requiring the wall or surface to be prepared to receive the material 10.

On the other hand, according to one aspect of the invention, it is advantageous to prepare products for receiving the application of the luminescent material 10. This preparation can include roughing the surface of a product (block 85) to provide enhanced capacity for adhesion of the luminescent material 10. Likewise, the product may be coated with a base material, such as a white base material, which may enhance the visibility of the luminescent material 10 in both light and dark conditions (block 90). Similarly, a product composed of a dark material, such as dark apparel, may be bleached prior to application of the material 10 (block 90). A plasti-charge ink which is pastisol-based may also be applied to the area receiving the luminescent material 10 to lighten dye coloration.

After the luminescent material is applied using one of the application methods described above, the luminescent material is left to dry (block 110). However, according to one aspect of the invention, the material 10 may be cured by a flash process exposing the material 10 to a quick burst of heat at a temperature of approximately 300 degrees Fahrenheit (block 100). This may be accomplished using a heating element such as a quartz lamp. Alternatively, a slower heating process can be utilized to speed up the curing of the luminescent material 10 (block 100).

As described above, the luminescent material 10 can be used to produce a wide variety of luminescent products which would benefit from enhanced visibility in the dark, including household and recreational items, such as paints, clothing, material, toys, cosmetics and the like, as well as outdoor articles such as paint, road signs, decals, and safety equipment. Some specific items producible using the luminescent material 10 include crayons, light bulbs, asthma inhalers, wall or outlet plates, interior and exterior paint, decals, toys, road signs, road reflectors, fingernail polish, and automobile finishes.

Many modifications and other embodiments of the inventions set forth herein will come to mind to one skilled in the art to which these inventions pertain having the benefit of the teachings presented in the foregoing descriptions and the associated drawings. Thus, it will be appreciated by those of ordinary skill in the art that the present invention may be embodied in many forms and should not be limited to the embodiments described above. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. A method of manufacturing a luminescent product that exhibits a daytime color and a glow in the dark color, where the glow in the dark color exhibits long term glow in the dark characteristics after absorbing energy from light sources, comprising:
mixing at least one resin material and at least one antisettling agent to produce a resin material and antisettling agent mixture;
adding at least one luminescent crystal to said resin material and antisettling agent mixture to produce a resin and luminescent crystal mixture;
mixing at least one solvent with at least one colorant to produce a solvent and colorant mixture; and
combining said resin and luminescent crystal mixture with said solvent and colorant mixture, and at least one additive, to produce a luminescent material, wherein said luminescent material exhibits a daytime color and a glow-in-the-dark color.

2. The method of claim 1, wherein said daytime color differs from said glow-in-the-dark color.

3. The method of claim 1, wherein the step of combining said resin and luminescent crystal mixture with said solvent and colorant mixture and at least one additive comprises the step of combining said resin and luminescent crystal mixture with said solvent and colorant mixture and at least one additive to produce a luminescent material, wherein said luminescent material comprises a paint or dye.

4. The method of claim 1, wherein said at least one additive comprises a plurality of microspheres, and wherein said plurality of microspheres provide an insulting effect to the luminescent material.

5. The method of claim 4, wherein said luminescent material comprises an insulating paint or dye.

6. The method of claim 1, wherein the step of combining said resin and luminescent crystal mixture with said solvent and colorant mixture and at least one additive comprises the step of combining said resin and luminescent crystal mixture with said solvent and colorant mixture and at least one additive to produce a luminescent material, wherein said luminescent material comprises luminescent tape.

7. The method of claim 1, wherein the step of combining said resin and luminescent crystal mixture with said solvent and colorant mixture and at least one additive comprises the step of combining said resin and luminescent crystal mixture with said solvent and colorant mixture and at least one additive to produce a luminescent material, wherein said luminescent material comprises at least 50%, by volume, of said at least one resin material.

8. The method of claim 1, wherein the step of mixing said at least one resin material and at least one antisettling agent comprises the step of mixing said at least one resin material and at least one antisettling agent using high speed dispersion or a static mixer.

9. The method of claim 1, further comprising the step of encapsulating said at least one luminescent crystal prior to the step of adding said at least one luminescent crystal to said resin material and antisettling agent mixture to produce a resin and luminescent crystal mixture.

10. The method of claim 1, further comprising the step of applying said luminescent material to an item to produce a luminescent product.

11. The method of claim 10, wherein the step of applying said luminescent material to said item comprises using at least one applicator to apply said luminescent material, wherein said at least one applicator is selected from the group consisting of: a paint brush, a foam brush, an airless sprayer, a paint gun, a screen printer, and a dot jet printer.

12. The method of claim 10, further comprising the step of heating said luminescent material after said luminescent material is applied to said item using said at least one applicator.

13. The method of claim 10, further comprising the step of priming the item with a base material prior to application of said luminescent material.

14. A luminescent product that exhibits a daytime color and a glow in the dark color, where the glow in the dark color exhibits long term glow in the dark characteristics after absorbing energy from light sources, comprising:
a luminescent material comprising:
at least one resin material;
at least one antisettling agent;
at least one colorant;
at least one additive; and
at least one luminescent crystal,
wherein said luminescent material exhibits a daytime color and a glow-in-the-dark color, and wherein said daytime color differs from said glow-in-the-dark color.

15. The product of claim 14, wherein said product further includes at least one base material in contact with the luminescent material.

16. The product of claim 14, wherein said product comprises nail polish.

17. The product of claim 14, wherein said product comprises fabric paint.

18. The product of claim 14, wherein said product comprises a product selected from the list of products consisting of: reflective tape, outlet covers, lamp shades and apparel.

19. The product of claim 14, wherein said luminescent material comprises a plurality of microspheres, wherein said plurality of microspheres provide an insulting effect to the luminescent material.

20. The product of claim 19, wherein said luminescent material comprises insulating paint.
